(19) United States
(12) Patent Application Publication
(10) Pub. No.: US 2020/0353128 A1
(43) Pub. Date: Nov. 12, 2020

(54) ANTIBIOTIC-GRAFTED ALUMINA CERAMIC MATRIX

(71) Applicant: I.CERAM, LIMOGES (FR)

(72) Inventors: André KERISIT, Limoges (FR); Evelyne POLI, Couzeix (FR); Guislaine BARRIÈRE, Saint Léonard de Noblat (FR)

(73) Assignee: I.CERAM, LIMOGES (FR)

(21) Appl. No.: 16/765,928

(22) PCT Filed: Nov. 28, 2018
(86) PCT No.: PCT/FR2018/053018

§ 371 Date: May 21, 2020

(30) Foreign Application Priority Data
Nov. 28, 2017 (FR) 1761317

(51) Int. Cl. A61L 27/54; A61L 27/56; C07K 9/00; A61L 27/42; C04B 38/00; C04B 41/00; C04B 41/45

(52) U.S. Cl. CPC A61L 27/54; A61L 27/56; C07K 9/008; A61L 27/427; C04B 38/0054; C04B 2235/3217; C04B 41/4556; A61L 2300/406; A61L 2430/02; C04B 2235/963; C04B 41/009

(57) ABSTRACT

The object of the invention is a porous alumina ceramic matrix grafted with an antibiotic, in particular vancoymicin, as well as its use in therapy. The invention also relates to a prosthesis or an implant using this matrix. The invention also relates to a method for preparing a matrix according to the invention.

### FIELD OF THE INVENTION

The present invention relates to the field of surgical implants, in particular those used as bone substitutes.

### TECHNICAL BACKGROUND

The occurrence of infections during surgical procedures, such as, in particular, the placement of implants, is one of the major concerns of the medical community.

The implants currently used in surgery are of various types: titanium, stainless steel, polyethylene, hydroxyapatite-based ceramic or others.

The conditions for the development of bacteria on the surface of the implants may be linked, in part, to the presence of nutrients under the influence of temperature, humidity, pH of the medium, as well as to the nature of the support. The surfaces of surgical implants may represent ideal substrates for bacterial colonization. The resulting infections are notorious for being difficult to treat, as well as the adhesion of bacteria, and the formation of biofilm that weakens the sensitivity to antibiotics.

Bacterial biofilms are structured clusters of bacterial cells coated with a polymer matrix and attached to a surface. Biofilm protects the bacteria and enables their survival in hostile environmental conditions. Biofilm bacteria can resist the host's immune response and are generally very resistant to antibiotics. Several microorganisms, such as Staphylococcus aureus or Pseudomonas aeruginosa, have this capacity to form a biofilm. The presence of biofilms during infections therefore calls for new methods of prevention and treatment.

The phase of the bacteria anchoring on the implant surface seems to be the most critical step in the development of peri-prosthetic infections.

In order to reduce this bacterial adhesion, it is known to chemically modify the metal surfaces of surgical implants. Thus, Antoci et al. (V. Antoci Jr. et al. Biomaterials 29 (2008) 4684-4690) and Kruszewski et al. (K. M. Kruszewski et al. Materials Science and Engineering C33 (2013) 2059-2069) respectively functionalized TA6V titanium and 316L stainless steel implants with vancomycin by performing a long multi-step synthesis.

Thus, Antoci describes a synthesis with the following steps: passivation (H₂O₂/H₂SO₄), aminopropylation with aminopropyl-triethoxysilane, coupling with 2 Fmoc-AEEA spacers, then coupling with vancomycin. Kruszewski describes a synthesis with the following steps: treatment with octadecylphosphonic acid, then with octacosanoic acid, with triethylene glycol with a phosphonic acid ending, then with pentaethylene glycol with a phosphonic acid ending, with 16-phosphonohexadecanoic acid, then coupling with vancomycin or gentamycin. This synthesis results in the formation of multilayers on the surface of the implant that immobilize the antibiotic; these layers are sometimes called SAM for Self-Assembled Monolayers.

There are also metallic nanoparticles coated with silica functionalized with vancomycin as described in Kell et al. (A. J. Kell et al. Chem. Commun., 2007, 1227-1229). In this publication, the silica coatings are treated to provide them with an amino or carboxylate ending, which may then bind to the carboxylic acid or amine function of the vancosamine function of vancomycin.

Mesoporous silica nanoparticles also functionalized with vancomycin have also been described by Qi et al. (ACS Appl. Mater. Interfaces, Publication Date (Web): 16 Oct. 2013). The mesoporous silica nanoparticles are functionalized by adding APS, then vancomycin in admixture with NHS and EDC.

These nanoparticles are clearly not suitable for use as an implant.

Also known are acrylic cements (PMMA type) releasing vancomycin. These are currently used by surgeons as a spacer for a short period of time, but have the disadvantage of having to be removed from the patient's body to make way for an implant. However, there is no coupling and thus no long-term protection.

Finally, Palchesko et al. (Materials Science and Engineering C31 (2011) 637-642) describes a method for coupling vancomycin on a calcium aluminum oxide support (calcium aluminate).

There is a need to further decrease the capacity for biofilm formation on the surface of an alumina ceramic implant.

### SUMMARY OF THE INVENTION

The invention relates firstly to a porous alumina ceramic matrix grafted with an antibiotic.

According to one embodiment, the porosity of the matrix is between 40 and 80%, preferably between 60 and 70%, advantageously about 65%.

According to one embodiment, the size of the pores is from 100 to 900 μm, preferably from 200 to 600 μm, more preferably about 400 μm.

According to one embodiment, the ceramic matrix has a volume porosity of 45 to 75%, a pore size of 100 to 900 μm, wherein the ceramic is obtained by impregnating a foam, pre-sintering at a temperature above 1200° C., superimpregnation with a slip, and sintering at a temperature above 1500° C., for example above 1600° C.

According to one embodiment, the antibiotic is chosen from among:
beta-lactamases, in particular amoxicillin, oxacillin, cloxacillin, ceftriaxone, cefotaxime, ceftazidime, piperacillin, imipenen, ertapenem, ceftaroline, aztreonam, cefepime, cefazolin;
clofazimine;
glycopeptides, in particular vancomycin, its derivatives, teicoplanin;
lipoglycopeptides, in particular dalbavancin, oritavancin, telavancin, daptomycin, preferably vancomycin or a derivative, and specifically vancomycin.

According to another aspect of the invention, the matrix according to the invention decreases and/or prevents bacterial proliferation and/or the formation of bacterial biofilm.

According to another aspect of the invention, the matrix is used in therapy or prophylaxis, in particular in the treatment of bone infections, bone cancers, in particular bone metastases.

According to one embodiment, the matrix is used in therapy as blocks of bone fillings, blocks of corpectomy, cages or intervertebral wedges of the cervical or lumbar spine, cervical wedges, calcaneus wedges, osteotomy wedges such as additional tibial wedges, derotation wedges such for anterior tibial tuberosity, compensation, valgization, reconstruction and filling blocks, pins, wedges and pivots for fixation and arthrodesis, drill bit pellets, arthrodesis blocks ensuring maintenance of the natural space or intersomatic interline, special reconstruction implants: sinus filling block, orbit floor and ceiling, craniotomy plugs, filling implants and maxillofacial rehab plates, and any anatomical part on which it is possible to reinsert “nerves” or “facia lata” tissues.

According to another aspect of the invention, the matrix is a prosthesis or an implant.

The subject of the invention is also a method for preparing a matrix according to the invention, comprising the following steps:
reaction of a diacid on the alumina ceramic matrix;
formation of an ester;
transamidification by reaction on an amino group of the antibiotic.

According to one embodiment, the ester is an N-hydroxysuccinimide ester, preferably formed in the presence of 1-ethyl-3-(3dimethylaminopropyl)carbodiimide.

The present invention overcomes the disadvantages of the prior art. More particularly, it provides a simple and effective means for reducing bacterial proliferation on the surface of an alumina ceramic.

This is accomplished through a method of coupling an antibiotic to an alumina ceramic matrix.

### BRIEF DESCRIPTION OF THE FIGURES

FIG. 1 represents vancomycin and the reactive amine site (vancosamine) thereof during grafting, and located opposite the effective part of the molecule that is represented with the interactions.

### DESCRIPTION OF EMBODIMENTS OF THE INVENTION

The invention is now described in more detail and in a non-limiting manner in the description below.

### Matrix

The matrix used in the present invention is a porous ceramic based on alumina Al₂O₃.

The (open) porosity of this ceramic may, in particular, be between 40 and 80%, preferably between 60 and 70%, advantageously about 65%

The pore size is typically from 100 to 900 μm, preferably from 200 to 600 μm, more preferably about 400 μm.

The porosity/pore size is measured by mercury porosimetry. The porosity is defined by the difference between the volume occupied by the pores and the total volume, wherein the total volume is the sum of the volume of the pores and of the alumina. The weight of alumina is defined by the volume and the density, wherein, by weighing the sample and knowing its total volume, one can determine by difference the volume of pores and therefore the (open) porosity.

The size of the ceramic matrix is variable and may range from a few millimeters to several centimeters or even tens of centimeters; the volume may be between 1 and 250 cm³, for example between 5 and 100 cm³.

The mechanical resistance to compression is advantageously between 20 and 60 MPa, advantageously greater than 40 MPa.

Any known method for the preparation of porous alumina may generally be used. One may, in particular, use a method comprising the following steps:
(A) supply of a pore-forming material (of the foam type, for example polyurethane foam, that is used, in particular, to regulate the porosity and the size of the pores) and impregnation of the pore-forming material with a suspension of alumina ceramic particles (alumina slip), possibly mixed with various organic additives such as binders, plasticizers and dispersants;
(B) drying in an oven;
(C) heat treatment at low temperature (below 700° C.) to remove the foam and organic constituents from the suspension; then
(D) sintering at a temperature above 1500° C., for example above 1600° C.

The method described in patent application FR2823674 may advantageously be used.

### Antibiotic Active Ingredient

The antibiotic to be grafted is an antibiotic having an accessible amine functional group and having an action on the bacterial wall. Antibiotics having an action on the wall on the bacteria are preferred to those having an action inside the bacteria.

Antibiotics may be chosen from the following non-exhaustive list:
beta-lactamases, in particular amoxicillin, oxacillin, cloxacillin, ceftriaxone, cefotaxime, ceftazidime, piperacillin, imipenen, ertapenem, ceftaroline, aztreonam, cefepime, cefazolin;
clofazimine;
glycopeptides, in particular vancomycin, its derivatives, teicoplanin;
lipoglycopeptides, in particular dalbavancin, oritavancin, telavancin, daptomycin.

The preferred antibiotic is vancomycin and its derivatives.

According to the present invention, the antibiotic is present on the surface of the ceramic matrix, wherein the surface may be the internal surface of the alumina ceramic. The antibiotic is grafted via covalent bonds.

### Grafting Method

The grafting method makes it possible to link the antibiotic via spacers with an amine functional group on the surface of the alumina ceramic.

The first step involves contacting a diacid in an appropriate solvent. The solvent may be tetrahydrofuran (THF). The diacid may be an aliphatic diacid comprising, apart from the acid functions, from 4 to 14 carbon atoms, for example 8, and may be 1,10-decanedicarboxylic acid. The excess solvent is removed by thorough evaporation. This first step results in the grafting of the spacers on the surface using two hydroxyl groups present on the surface.

The second step is the formation of an N-hydroxysuccinimide (NHS) ester. The acid-modified ceramic is placed in a solution of N-hydroxysuccinimide (NHS) with 1-ethyl-3-(3-dimethylaminopropyl)carbodiimide (EDC), in a buffer, for example, of 2-(N-morpholino)ethanesulfonic (MES) acid, for example at room temperature for a time between 10 minutes and 2 hours. The EDC/NHS combination is known for the binding of biomolecules. An NHS ester is obtained at the end of this second step.

The third step is the coupling of the antibiotic. An accessible amine function, based on the application of the principles of steric hindrance, will then react with the NHS ester to form an amide bond, by transamidation. The reaction solvent may be the MES buffer or an organic solvent such as ethanol. The reaction temperature may be, for example, at room temperature. The reaction time may be, for example, between 1 and 48 hours. The pellets are then washed, for example using the same solvent, then cleaned and dried in the conventional manner.

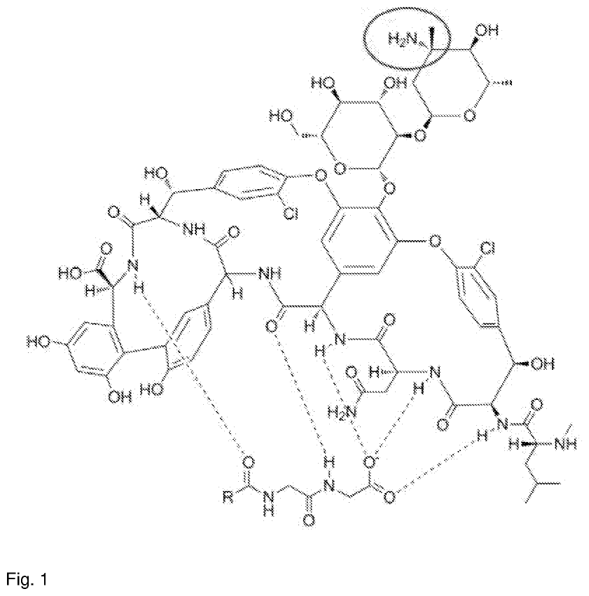

In the case of vancomycin, the reactive site is the glucose-related vancosamine function. This reactive part is identified in FIG. 1, with the indication of the interactions between vancomycin and the D-ala-D-ala residues of the peptidoclycan of the bacterial wall.

### Applications.

The invention finds application in a large number of uses, in particular in traumatology for reconstructions of fractures, in orthopedics as filling elements, or as implants (for fusion of the spine, etc.), for osteotomies through addition, and for reconstructions, (for example maxillofacial) and, in particular, for interventions in the context of treatment of bone cancers, of a primary or secondary nature.

The invention allows the production of numerous bone substitutes and implants, which may be used, for example, as addition wedges or for bone filling throughout the entire skeleton.

Exemplary applications may be cited such as bone filling blocks, corpectomy blocks, cages or intersomatic wedges of the cervical or lumbar spine, cervical wedges, calcaneus wedges, osteotomy wedges (e.g. tibial addition wedges), derotation wedges (e.g. anterior tibial tuberosity), compensation, valgization, reconstruction and filling blocks, fixing and arthrodesis wedges and pivots, drill bit pellets, arthrodesis blocks ensuring maintenance of the natural space or intersomatic interline, special reconstruction implants: sinus filling block, orbit floor and ceiling, craniotomy plugs, bone filling implants whatever the location of the skeleton, and generally any anatomically-shaped part on which it is possible to reinsert “nerves” or “facia lata” tissues wherever the location of implantation on the skeleton.

The grafting of the antibiotic such as vancomycin on the ceramic reduces the risk of colonization of the material by germs sensitive to vancomycin. The grafted vancomycin interacts with the D-ala-D-ala produced by the bacteria and prevents bacterial transpeptidases (penicillin-binding protein) from forming the peptidoglycan. Therefore, the bacteria cannot form its wall and is lysed.

A particular interest of the invention is long-term protection of the implant to avoid colonization (i) during implantation but, also, (ii) over time given the covalent grafting of the molecule.

### EXAMPLE

The following example illustrates the invention without limiting it.

Step 1:

Alumina ceramic pellets were placed in a solution of 1,10-decanedicarboxylic acid in dry THF (tetrahydrofuran) at room temperature. The excess solvent was removed by placing in the oven.

This first step results in the grafting of the spacers on the surface using two hydroxyl groups present on this surface.

Step 2:

The pellets modified by 1,10-decanedicarboxylic acid were then placed in a solution of 1-ethyl-3-(3-dimethylaminopropyl) carbodiimide (EDC)/N-hydroxysuccinimide (NHS) in a 2-(N-morpholino)ethanesulfonic (MES) acid buffer at room temperature to obtain an NHS ester.

Step 3:

Finally, the vancomycin was allowed to react with the pellets from the previous step in the MES buffer. The pellets were then washed with MES buffer and cleaned in an ultrasonic bath.

FIG. 1 shows the vancomycin molecule with the reaction amine, vancosamine.

1.-12. (canceled)

13. A porous alumina ceramic matrix grafted with an antibiotic.

14. The matrix according to claim 13, wherein the porosity of the matrix is between 40 and 80%.

15. The matrix according to claim 13, wherein the pore size is from 100 to 900 μm.

16. The matrix according to claim 13, wherein the porosity is open.

17. The matrix according to claim 13, wherein the mechanical resistance to compression is between 20 and 60 MPa.

18. The matrix according to claim 13, wherein the ceramic matrix has a volume porosity of 45 to 75%, and a pore size of 100 to 900 μm, wherein the ceramic is obtained by impregnation of a foam, pre-sintering at a temperature above 1200° C., superpregnation with a slip, and sintering at a temperature above 1500° C.

19. The matrix according to claim 13, wherein the antibiotic is chosen from:
beta-lactamases, in particular amoxicillin, oxacillin, cloxacillin, ceftriaxone, cefotaxime, ceftazidime, piperacillin, imipenen, ertapenem, ceftaroline, aztreonam, cefepime, cefazolin;
clofazimine;
glycopeptides, in particular vancomycin, its derivatives, teicoplanin; and
lipoglycopeptides, in particular dalbavancin, oritavancin, telavancin, daptomycin.

20. The matrix according to claim 19, wherein the antibiotic is vancomycin or a derivative.

21. The matrix according to claim 13, for reducing and/or preventing bacterial proliferation and/or the formation of bacterial biofilm.

22. The matrix according to claim 13, for use in therapy or prophylaxis.

23. The matrix according to claim 13, for use in the treatment of bone infections or bone cancers.

24. The matrix according to claim 13, for use in therapy as blocks of bone fillings, blocks of corpectomy, cages or intersomatic wedges of the cervical or lumbar spine, cervical wedges, calcaneus wedges, osteotomy wedges such as the addition of tibial wedges, derotation wedges such as anterior tibual tuberosity, compensation, valgization, reconstruction and filling blocks, pins, fixation and arthrodesis wedges and pivots, drill pellets, arthrodesis blocks ensuring the maintenance of natural space or intersomatic interline, special reconstruction implants: sinus filling block, orbit floor and ceiling, craniotomy plugs, filling implants and maxillofacial rehab plates, and any anatomical part on which it is possible to reinsert “nerves” or “facia lata” tissues.

25. The matrix according to claim 13 forming a prosthesis or an implant.

26. A method of preparing a porous alumina ceramic matrix grafted with an antibiotic, comprising the following steps:
reaction of a diacid on the alumina ceramic matrix;
formation of an ester; and
transamidification by reaction on an amino group of the antibiotic.

27. The method of preparing a matrix according to claim 26, wherein the ester is an N-hydroxysuccinimide ester.

28. The method of preparing a matrix according to claim 26, wherein the ester is formed in the presence of 1-ethyl-3-(3-dimethylaminopropyl)carbodiimide.
